(19) United States
(12) Patent Application Publication
(10) Pub. No.: US 2008/0312528 A1
(43) Pub. Date: Dec. 18, 2008

(54) GUIDANCE OF MEDICAL INSTRUMENT USING FLOUROSCOPY SCANNER WITH MULTPLE X-RAY SOURCES

(76) Inventor: James A. Bertolina, Portage, MI (US)

Correspondence Address: CARLSON, GASKEY & OLDS, P.C., 400 WEST MAPLE ROAD, SUITE 350, BIRMINGHAM, MI, 48009, US

(21) Appl. No.: 12/139,730

(22) Filed: Jun. 16, 2008

Related U.S. Application Data
(60) Provisional application No. 60/944,234, filed on Jun. 15, 2007.

(51) Int. Cl. A61B 6/00

(52) U.S. Cl. 600/426; 382/132

(57) ABSTRACT

A scanner takes an initial image of a patient, and a known structure in the initial image is identified. Based on the initial image, a location for injection of a chemical with a medical instrument can be determined. During a medical procedure, the scanner operates in a fluoroscopy mode, and real-time images of the patient are displayed. The medical instrument includes two markers having a fixed and known relationship and orientation relative to each other and a tip of the medical instrument Movement of the tip is monitored as it is inserted into the patient, and the location of the tip relative to the injection location is tracked based on the detected relationship of the markers of the medical instrument and the known structure. When the computer determines that the tip is at the injection location, the computer provides an alert to inject the chemical.

### REFERENCE TO RELATED APPLICATIONS

This patent application claims priority to U.S. Provisional Application No. 60/944,234 filed on Jun. 15, 2007.

### BACKGROUND OF THE INVENTION

The present invention relates generally to a scanning system used to track a medical instrument for precise injection of a chemical into an injection location of a patient.

During a medical procedure, a chemical may need to be injected in a patient. The location of the injection must be precisely located to ensure that the chemical is injected at the proper location in the patient.

### SUMMARY OF THE INVENTION

A scanning system includes a scanner having two x-ray sources. Prior to a medical procedure, the scanner takes an initial image of the patient. A known structure in the initial image is identified. Based on the initial image, an injection location of a chemical into the patient can be determined relative to the known structure.

During a medical procedure, a chemical is injected at the injection location through a tip of a medical instrument. The medical instrument includes at least two markers having a fixed and known relationship and orientation relative to each other and the tip of the medical instrument. The scanner operates in a fluoroscopy mode, and real-time images of the patient and the markers are shown on a display. The computer combines the images obtained from each of the x-ray sources to create one displayed image. Each x-ray source is automatically collimated to specifically direct the x-rays towards the injection location and the medical instrument, preventing additional x-ray exposure to the patient.

The relationship between the tip and the markers of the medical instrument is known, and the relationship between the known structure and the injection location is known. The computer monitors the movement of the tip as it is inserted into the patient and tracks the location of the tip relative to the injection location based on the detected relationship of the markers of the medical instrument and the known structure. When the computer determines that the tip is located at the injection location, the computer provides a signal to the surgeon. The surgeon then injects the chemical into the injection location using the medical instrument.

These and other features of the present invention will be best understood from the following specification and drawings.

### BRIEF DESCRIPTION OF THE DRAWINGS

The various features and advantages of the invention will become apparent to those skilled in the art from the following detailed description of the currently preferred embodiment. The drawings that accompany the detailed description can be briefly described as follows:

FIG. 1 schematically illustrates a scanning system of the present invention.

### DETAILED DESCRIPTION OF THE PREFERRED EMBODIMENT

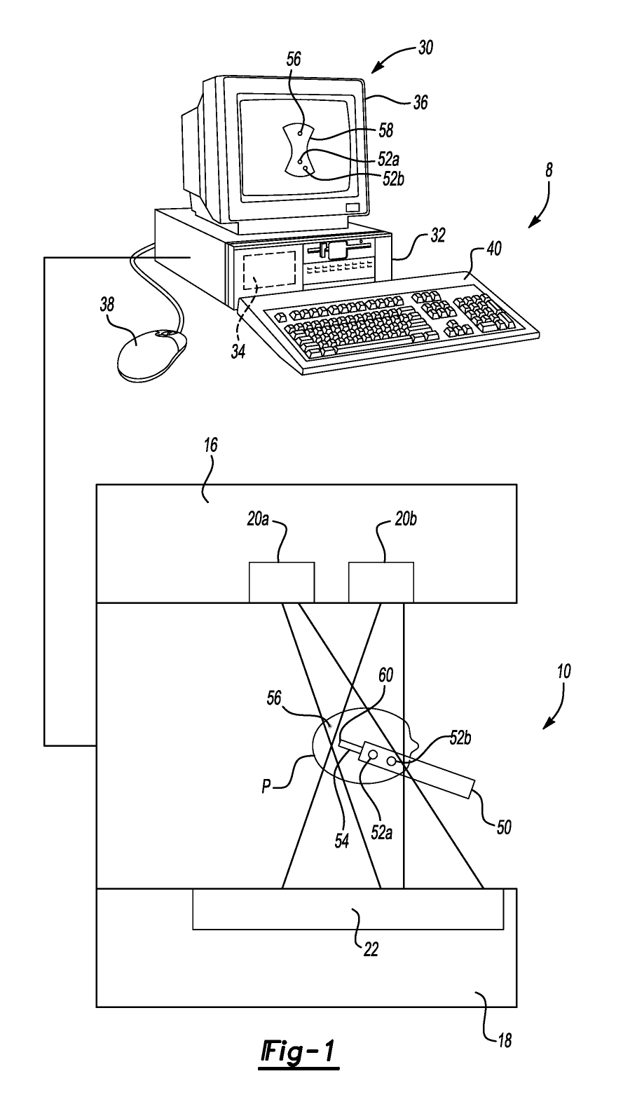

FIG. 1 illustrates a scanning system 8 including a gantry 12 that supports and houses components of a scanner 10. Suitable scanners 10 are known. In one example, the scanner 10 can be a CT scanner or an MRI scanner. In one example, the scanner 10 is an x-ray scanner.

The gantry 12 includes a first arm 16 and a second arm 18. The first arm 16 houses two x-ray sources 20a and 20b that generate x-rays 28. In one example, the x-ray sources 20a and 20b are cone-beam x-ray sources. The second arm 18 houses a complementary flat-panel detector 22 spaced apart from the x-ray sources 20a and 20b. The x-rays 28 are directed toward the detector 22 which includes a converter (not shown) that converts the x-rays 28 from the x-ray sources 20a and 20b to visible light and an array of photodetectors behind the converter to create an image. Various configurations and types of x-ray sources 20a and 20b and detectors 22 can be utilized, and the invention is largely independent of the specific technology used for the scanner 10.

The scanner 10 further includes a computer 30 having a microprocessor or CPU 32, a storage 34 (memory, hard drive, optical, and/or magnetic, etc), a display 36, a mouse 38, a keyboard 40 and other hardware and software for performing the functions described herein. A plurality of images 58 taken by the detector 22 are sent to the computer 30. The images 58 are stored on the storage 34 of the computer 30 and can be displayed on the display 36 for viewing.

Prior to a medical procedure, the scanner 10 takes an initial image (CT or MRI) of the patient P that is stored by the computer 30. A known structure 56 in the initial image is identified. The known structure 56 can be a marker or markers positioned on the patient P (such as a metal BB) or a known specific structure in the patient's P anatomy. Based on the initial image, an injection location 60 for injection of a chemical (for example, a drug) can be determined. For example, the injection location 60 can be determined relative to the known structure 56. The injection location 60 is stored on the computer 30.

During the medical procedure, the chemical is injected in the patient P at the injection location 60. The chemical is injected through a tip 54 of a medical instrument 50. The medical instrument 50 includes at least two markers 52a and 52b. In one example, the markers 52a and 52b are metal BBs. In another example, the markers 52a and 52b are radio-opaque stripes. The markers 52a and 52b have a fixed and known relationship and orientation relative to each other and the tip 54 of the medical instrument 50.

During the medical procedure, the scanner 10 operates in a fluoroscopy mode, and real-time images 58 of the patient P and the markers 52a and 52b are shown on the display 36. The image associated with each of the x-ray sources 20a and 20b is stored by the computer 30, and the computer 30 combines the images obtained from each of the x-ray sources 20a and 20b to create one image 58 that is displayed on the display 36. That is, a portion of the images taken from each of the x-ray sources 20a and 20b overlap to create the displayed image 58.

X-rays 28 from both of the x-ray sources 20a and 20b are directed towards the injection location 60 and the medical instrument 50. Each x-ray source 20a and 20b is automatically collimated to specifically direct the x-rays towards the injection location 60 and the markers 52a and 52b of the medical instrument 50, preventing additional x-ray exposure to the patient P. The x-ray sources 20a and 20b are collimated such that the markers 52a and 52b are included in the image 58. If the computer 30 determines that one of the markers 52a and 52b are not visible in the image 58, the computer 30 then sends a signal to the x-ray source 20a and 20b to re-collimate the x-rays 28 until both the markers 52a and 52b are visible inside the images 58.

The relationship between the tip 54 and the markers 52a and 52b of the medical instrument 50 is known, and the relationship between the known structure 56 and the injection location 60 in the patient P is known. The computer 30 can detect the markers 52a and 52b and the known structure 56 in the images 58. Therefore, the computer 30 can monitor the movement of the tip 54 as it is inserted into the patient P and track the location of the tip 54 relative to the injection location 60 based on the information obtained in the images 58 and the relationship detected by the computer 30 between the markers 52a and 52b of the medical instrument 50 and the known structure 56.

For example, the computer 30 monitors the location of the markers 52a and 52a relative to the known structure 56 in the images 58 as the tip 54 moves. The location of the tip 54 relative to the injection location 60 can therefore be determined and monitored. A surgeon can also monitor the movement of the medical instrument 50 by observing movement of the markers 52a and 52b in the images 58 shown on the display 36 as the tip 54 is inserted into the patient P. The known structure 56 can also be visible in the images 58. When the computer determines that the tip 54 is located at the injection location 60 based on the known relationships, the computer 30 can provide a signal to the surgeon that the tip 54 is at the desired location. The surgeon then injects the chemical into the injection location 60 of the patient P using the medical instrument 50.

It is possible that both the markers 52a and 52b may not be visible in the image 58 at a given time based on the location and orientation of the medical instrument 50. That is, the medical instrument 50 could be oriented such that the markers 52a and 52b are aligned, preventing viewing and detection of both of the markers 52a and 52b in the image 58. If this occurs, the computer 30 generates an alarm to indicate that the orientation of the markers 52a and 52b relative to each other cannot be determined. The alarm can be visual or audio or any type of alarm. The medical instrument 50 can then be repositioned. Once the medical instrument 50 is oriented into a new position where the orientation of the markers 52a and 52b can be determined, the medical procedure can continue.

The foregoing description is only exemplary of the principles of the invention. Many modifications and variations are possible in light of the above teachings. It is, therefore, to be understood that within the scope of the appended claims, the invention may be practiced otherwise than using the example embodiments which have been specifically described. For that reason the following claims should be studied to determine the true scope and content of this invention.

What is claimed is:

1. A method of positioning a medical instrument, the method comprising the steps of:
obtaining an initial image of a patient;
identifying a known structure in the initial image;
determining an injection location for injection of a chemical into the patient relative to the known structure;
obtaining a plurality of images of the patient with a scanner, wherein the scanner includes two x-ray sources; and
monitoring a location of a tip of a medical instrument relative to the injection location by monitoring a relationship between at least two markers of the medical instrument and the known structure, wherein the at least two markers have a fixed and known relationship and orientation relative to each other and the tip.

2. The method as recited in claim 1 including the step of injecting the chemical at the injection location through the tip of the medical instrument.

3. The method as recited in claim 1 including the step of storing the initial image and the plurality of images.

4. The method as recited in claim 1 including the step of displaying the initial image and the plurality of images.

5. The method as recited in claim 1 wherein the at least two markers of the medical instrument are metal BBs or radio-opaque stripes.

6. The method as recited in claim 1 including the step of identifying the known structure with a marker.

7. The method as recited in claim 1 including the step of operating the scanner in a fluoroscopy mode during the steps of obtaining and monitoring.

8. The method as recited in claim 1 including the step of combining an image associated with each of the two x-ray sources to create a single image that is employed to generate the plurality of images.

9. The method as recited in claim 1 including the step of automatically collimating both of the two x-ray sources to specifically direct the x-rays towards the injection location and the at least two markers of the medical instrument.

10. The method as recited in claim 1 including the step of providing a signal if all of the at least two markers of the medical instrument are not visible in the plurality of images.

11. The method as recited in claim 1 including the step of providing a signal when the tip of the medical instrument is located at the injection location.

12. The method as recited in claim 1 wherein the steps of observing and monitoring occur simultaneously.

13. A scanning system comprising:
a medical instrument including a tip and at least two markers having a fixed and known relationship and orientation relative to each other and the tip;
a scanner including two x-ray sources that obtains obtain a plurality of images of a patient; and
a computer to identify a known structure in the initial image, to determine an injection location for injection of a chemical into the patient relative to the known structure, and to monitor a location of the tip of the medical instrument relative to the injection location by a relationship between the at least two markers and the known structure.

14. The scanning system as recited in claim 13 wherein the scanner includes a gantry having a first arm and a second arm, and the first arm houses the two x-ray sources and the second arm houses a detector that converts the x-rays to create the initial image and the plurality of images.

15. The scanning system as recited in claim 13 wherein the computer stores the initial image and the plurality of images.

16. The scanning system as recited in claim 13 including a display that displays the initial image and the plurality of images.

17. The scanning system as recited in claim 13 wherein the at least two markers of the medical instrument are metal BBs or radio-opaque stripes.

18. The scanning system as recited in claim 13 including a marker located at the known location.

19. The scanning system as recited in claim 13 wherein the computer combines an image associated with each of the two x-ray sources to create a single image that is employed to generate the plurality of images.

20. The scanning system as recited in claim 13 wherein both of the two x-ray sources are collimated to specifically direct the x-rays towards the injection location and the at least two markers of the medical instrument.

21. The scanning system as recited in claim 13 wherein the computer provides a signal if all of the at least two markers of the medical instrument are not visible in the plurality of images.

22. The scanning system as recited in claim 13 wherein the computer provides a signal when the tip of the medical instrument is located at the injection location.
